(19) United States
(12) Patent Application Publication
(10) Pub. No.: US 2015/0094774 A1
(43) Pub. Date: Apr. 2, 2015

(54) BONE FIXATION ASSEMBLY, INCLUDING S-SHAPED RESILIENT LOCK FOR SCREW LOCKING CLIPS

(71) Applicants: Karl W. Swann, San Antonio, TX (US); Alliance Partners LLC, San Antonio, TX (US)

(72) Inventors: Karl W. Swann, San Antonio, TX (US); Frank Morris, Austin, TX (US); Andrew Enke, San Antonio, TX (US)

(21) Appl. No.: 14/504,955

(22) Filed: Oct. 2, 2014

Related U.S. Application Data
(60) Provisional application No. 61/885,763, filed on Oct. 2, 2013.

(51) Int. Cl. A61B 17/80

(52) U.S. Cl. CPC A61B 17/8038
USPC 606/291

(57) ABSTRACT

A bone plate assembly for stabilizing adjacent bones, the bone plate assembly having screw bores therein. The screws are dimensioned to sit within the screw bore and a resilient “S” shaped arm is engaged to the plate and partially covers the screw bore until contacted by the head of the screw. Contact with the head of the screw while the screw is being advanced into the bone pushes the resilient arm away from the head and, when the screw is seated in the screw bore, the resilient arm springs back to at least partially block the top of the screw head. In this manner, the screw is prevented form backing out of the plate.

This application claims the benefit of and priority to provisional patent application Ser. No. 61/885,763, filed Oct. 2, 2013, and incorporates the same herein by reference.

### FIELD OF THE INVENTION

Medical devices, more specifically, a bone fixation assembly for attachment between adjacent bones for stabilization, including locking mechanisms for screws of the bone fixation assembly.

### BACKGROUND OF THE INVENTION

Bone fixation assemblies having a plate or plates and screws typically incorporate locking mechanisms to prevent a screw or anchor from backing out, once the screw or anchor affixes the plate to the bone. These back out prevention systems typically use a variety of configured members to cover or otherwise engage the screws to prevent backout.

### SUMMARY OF THE INVENTION

The present invention provides an improved bone fixation assembly that, in one embodiment, incorporates a novel screw locking mechanism. The invention, in one embodiment, comprises a bone fixation plate having an upper surface and a bottom or bone contacting surface, and at least one screw bore with bore side walls extending through the plate. The assembly includes walls configured to receive, in one embodiment, a resilient S-shaped member or arm in a first position being both the rest and the locking position, and a second, screw head engaging expanded position.

In one embodiment, a bone fixation assembly may comprise a plate having an upper surface, a lower, bone contacting surface, an outer perimeter and a multiplicity of screw receiving bores therethrough. The screw bores have bore side walls. A multiplicity of screws may be provided, each screw having a threaded section with a threaded distal tip and a head at the opposite, proximal end from the tip. The threaded section of the screw meets the head. The head may have outer walls defining a conical section.

A multiplicity of resilient retainer arms is provided, in one embodiment, each having a curved, head engaging or collar section with a radius of curvature about equal or slightly greater than the maximum radius of curvature of the head top surface and the radius of curvature of the upper screw bore.

The upper surface of the plate may have a multiplicity of retainer arm recesses, the retainer arm recesses having a recess floor and recess side walls. The screw bores of the plate may each open into separate arm retaining recesses, the arms for engaging the arm retainer recesses, wherein the arm retainer recesses include pockets dimensioned to at least partly overhang the curved screw head engaging section. The arms may include means to engage or couple the arms to the arm retainer recesses, such that the arms do not separate from the plate.

A bone fixation assembly comprising a multiplicity of screws, each having a threaded section with a tip at a first end and a head having an upper surface at a second end, the head having a first diameter where it meets the threaded section and a larger diameter at the upper surface; and a head height between the two diameters; a plate having a plane and having an upper surface, a lower surface, and a multiplicity of screw bores therethrough, the screw bores having an upper opening having a first diameter and a lower opening having a second diameter and a screw bore height between the two diameters, the plate having a retainer arm recess floor defining a retainer arm recess floor plane, the retainer arm recess floor above the walls defining the screw bore, the plate having a multiplicity of pin bores therethrough, such that each screw bore has a pin bore adjacent thereto; and a single resilient retainer arm with a first end having a depending pin for engaging the pin bore of the plate adjacent each screw bore and a removed end with a body therebetween, the removed end not attached to the plate and free to move in the plane of the retainer arm recess floor such that in a first position part of the body blocks some of the upper opening of the screw bore and in a second position, responsive to contact with a screw head as the screw head rotates and moves longitudinally into a screw bore, moves to a second, loaded position outward from the first position, and with further longitudinal movement of the screw such that the upper surface of the screw head passes below the upper opening of the screw bore, substantially returns to the first position, wherein the screw bore height is at least about equal to the screw head height, wherein the retainer arm recess floor is below the upper surface of the plate.

The plate may include a pocket dimensioned to receive at least part of the resilient retainer arm, the pockets below the upper surface of the plate and above the screw bores, wherein the pocket may be dimensioned to receive part of the resilient retainer arm when the resilient retainer arm is moving towards and away from the loaded position, wherein the retainer arm is resilient and includes a lower surface which lays against the retainer arm recess floor and an intermediate surface between the upper and lower surface. The retainer arm may have an “S” shape.

The plate may include upstanding walls adjacent the first end which are dimensioned to contact retainer arm walls adjacent the first end while the resilient retainer arm is moving between the two positions, but not when the resilient retainer arm is in the first position. The plate, retainer arm, and screw are typically all in contrasting colors.

The screw head includes tool engaging walls, the tool engaging walls project upward from the upper surface of the screw head, wherein below the first diameter of the screw head, there are, in one embodiment, cylindrical walls perpendicular to the screw head with a diameter just slightly smaller than the second diameter of the plate and, in another embodiment, the screw head first diameter is substantially smaller than the second diameter of the plate allowing some angular movement of the screw with respect to the plate.

### BRIEF DESCRIPTION OF THE DRAWINGS

FIG. 1 is a perspective view of Applicant's bone fixation assembly with the screws inserted and in a first screw locked or rest position.

FIG. 1A is a cross-sectional view of one embodiment a screw used in Applicant's bone fixation assembly.

FIG. 2 is a view of FIG. 1, with some of the screws exploded away.

FIG. 2A is a perspective view of the plate with two of the resilient retainer arms exploded away.

FIG. 2B is a top view of a resilient arm apart from the device.

FIGS. 2C and 2D are cross-sectional views through the screw plate showing the groove or pocket in which the retainer arm may be at least partly located.

FIG. 3 is a perspective view of an embodiment of Applicant's assembly without screws, but with the retainer arm.

FIGS. 3A and 3B illustrate bottom and top views, respectively, of a screw bore of the plate without the screw in the screw bore and showing the position of the resilient retainer arm at rest and as it would be in a locking position.

FIG. 4 is a perspective cutaway view with the screws engaged to the plate and fully seated in a fully seated, locked down position. Detail A is a cross-section of part of the retainer arm in recessed portion of the assembly showing the position of the retainer arm with respect to the plate.

FIGS. 4A and 4B are top and side cutaway views of the assembly just before screw insertion.

FIGS. 4C and 4D are top and side cutaway views of the assembly during screw insertion.

FIGS. 4E and 4F are top and side cutaway views of the screw after insertion.

FIG. 4G is a top view of the plate with retainer arms, but no screws.

FIG. 4H is a side cutaway showing engagement of the retainer arm with respect to the plate.

FIG. 5 is a perspective view of the plate without screws, but with the retainer arms.

FIG. 6 is a perspective view of the underside of the plate without screws and without retainer arms.

FIGS. 7A, 7B, and 7C illustrate the use of an insertion tool for inserting the screws into the bone.

FIG. 8 illustrates a cross-sectional cutaway in perspective of the plate being used to stabilize adjacent vertebrae.

FIG. 9A and 9B illustrate a fixed pitch and variable pitch screw, respectively.

FIG. 10 illustrates a variable pitch screw in a plate, in cross-section.

### DETAILED DESCRIPTION OF THE PREFERRED EMBODIMENT

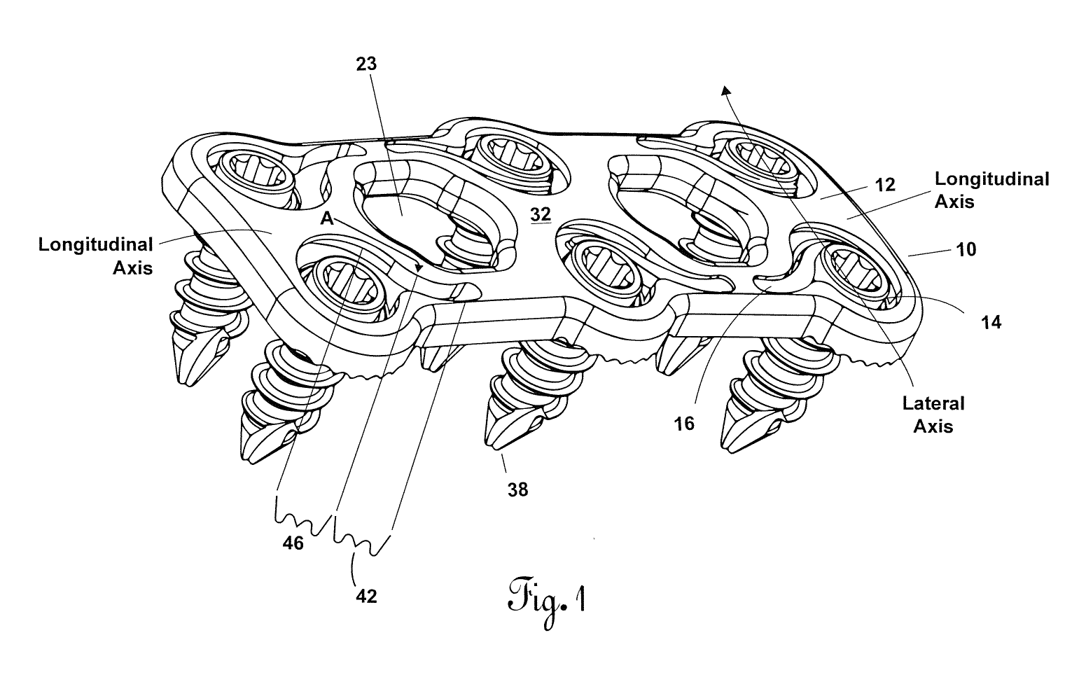

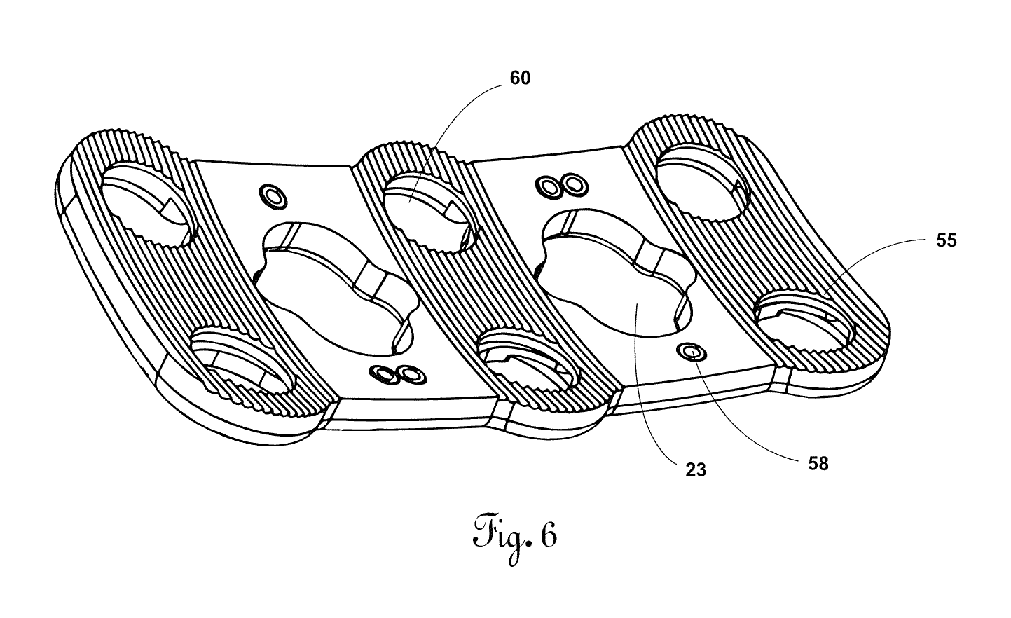

FIGS. 1 through 6 illustrate a bone fixation assembly 10 comprising a plate 12, a multiplicity of screws 14, and a multiplicity of retainer arms 16. Plate 12 is configured to lay adjacent a bone surface, the screws being received into the bone and plate and affixing plate 12 to the bone and the clips for lockingly engaging the screws to prevent the screws from backing out. Plate 12 may be slightly curved in an elevational side view to match the anterior curve of the spine.

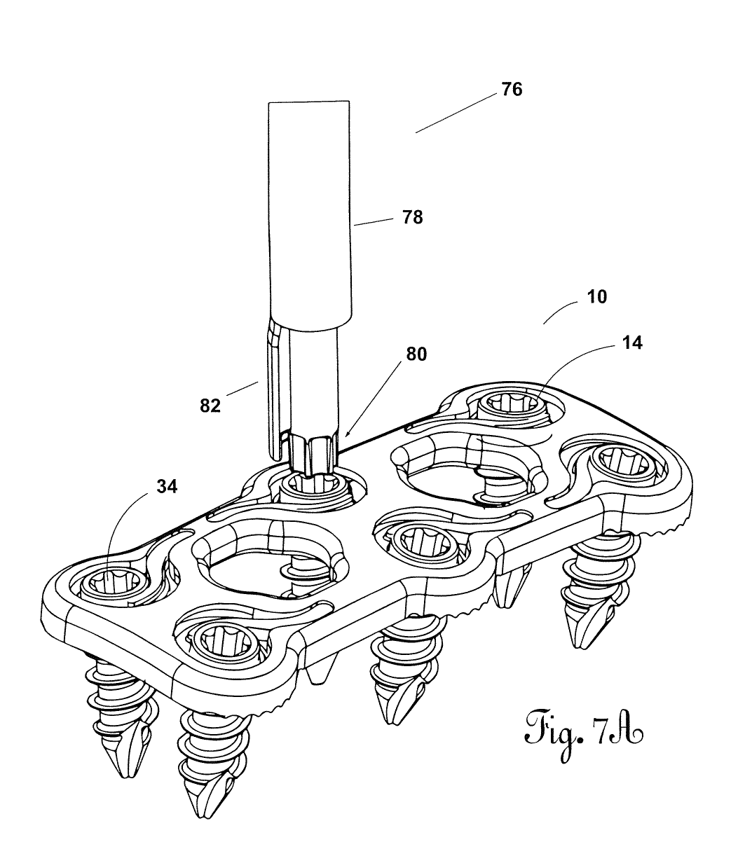

More specifically, the figures illustrate a plate 12 having an upper surface 18 thereon, a lower surface 20 (which may be smooth or serrated, see FIG. 6), and an outer perimeter 22 between the upper and lower surfaces. The plate may also have through or graft window opening or openings 23 therethrough, between the upper and lower surfaces.

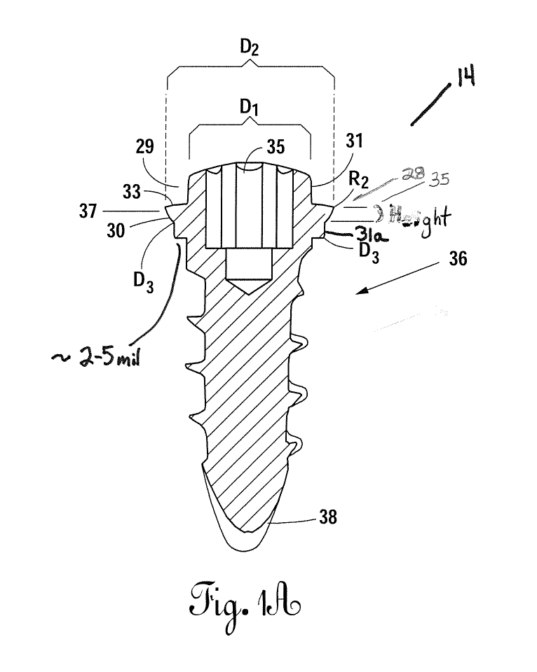

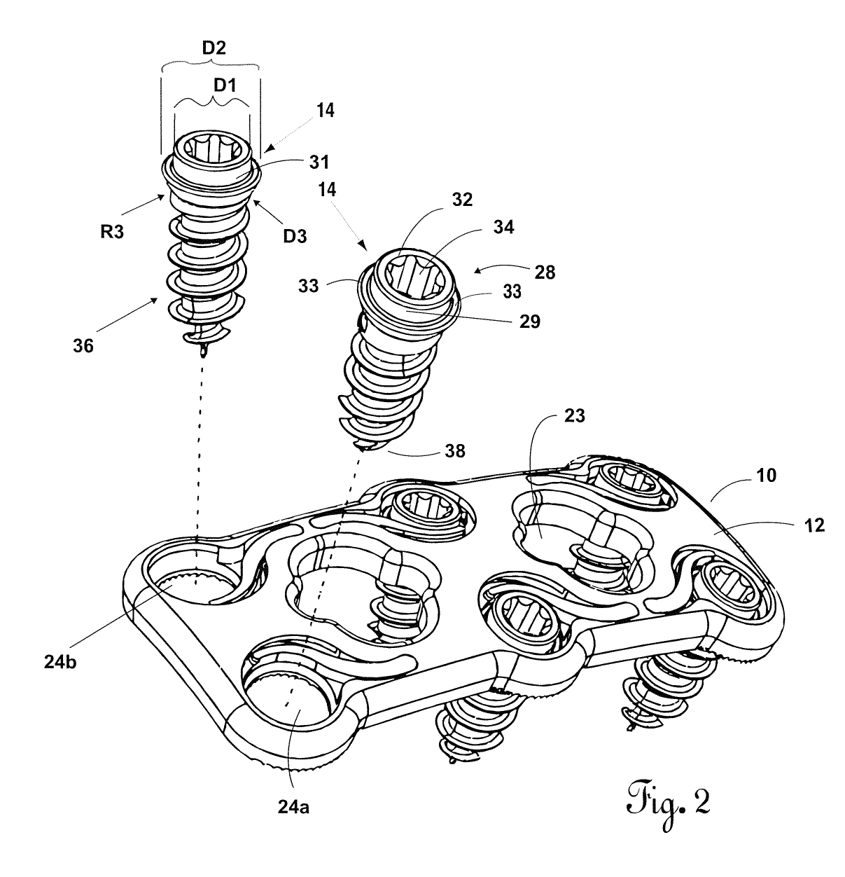

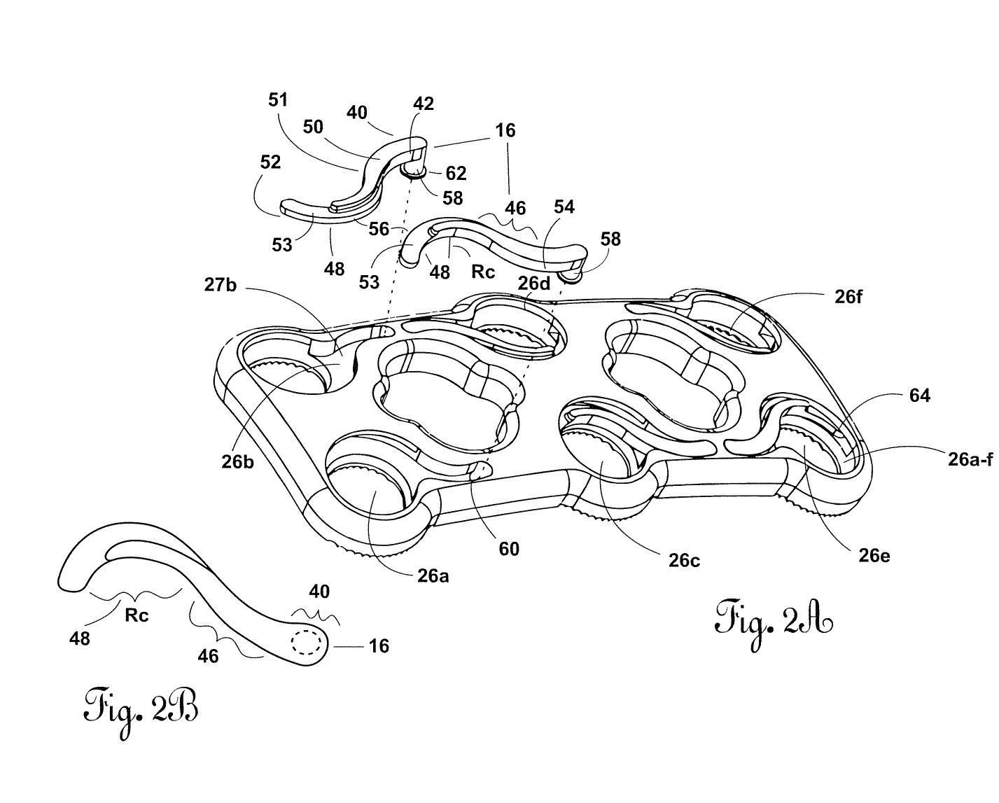

A multiplicity of screw bores 24a/24b/24c/24d/24e/24f may be provided. These may be paired and spaced laterally apart, and they are preferably four (one level), six (two level) or eight (three level) in number. Screw bores 24a/24b/24c/24d/24e/24f (two level) are seen to extend through the plate, the bores for receiving the screws, the screws to retain the plate against the bone. Each bore has a bore side wall 25, which is fully circumferential (see FIGS. 2B and 2C), which is shaped to receive a screw head 28 of a screw 14. The bore side walls have an upper opening with a first diameter and a lower opening with a second diameter, and the screw heads fit snugly but not necessarily tight between the two openings.

Upper surface 18 is seen to be configured with a multiplicity of clip retainer recesses 26, the clip retainer recesses at least partly surrounding and above the screw bores and having side walls 27a and arm retainer floors 27b (see FIG. 2A).

Screws 14 (see FIG. 1A and FIG. 2) are seen to include head 28 at the proximal end and a threaded section 36. Head 28 may be at least partly generally conical with an outward trending walls and threaded section 36 may be helical, ending in one embodiment at a distal bone cutting tip 38. Stepped back section 29 may, in one embodiment, include step side walls 31 and a head floor 33. Head floor 33 separates the conical section 35 from the stepped back section 29. Head 28 has side walls 30 defining a conical section 35 thereon. Head 28 may also include an upper surface 32, which may include tool engaging, typically recessed, walls 34 configured for receipt of a tool therein.

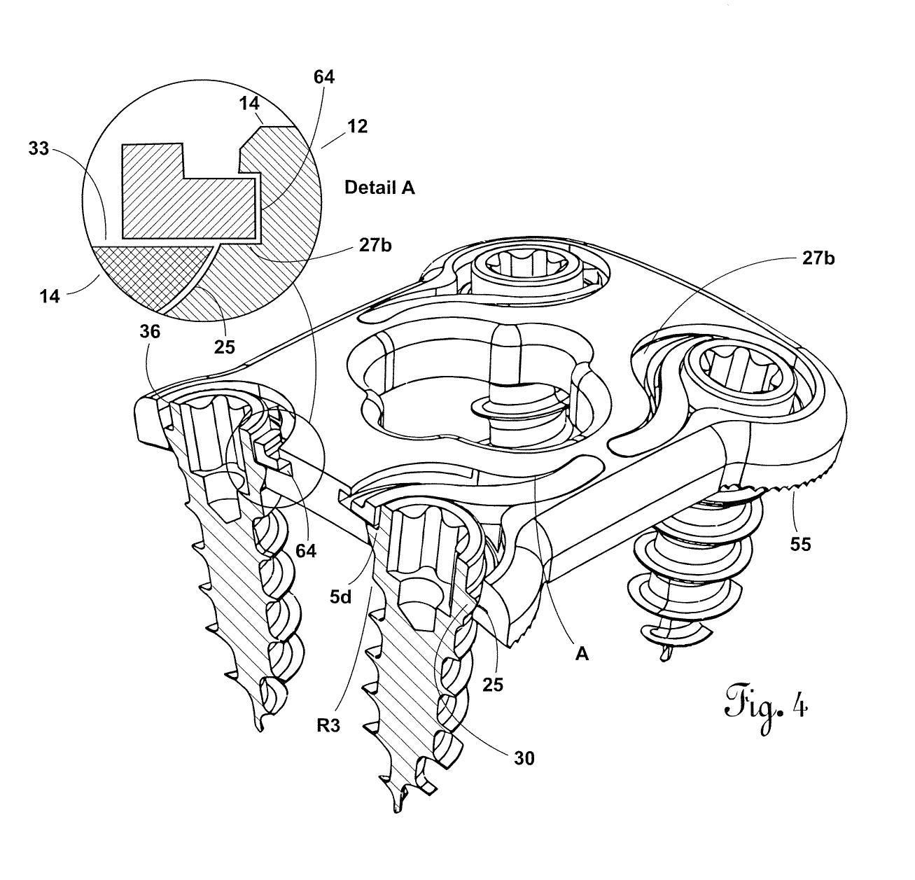

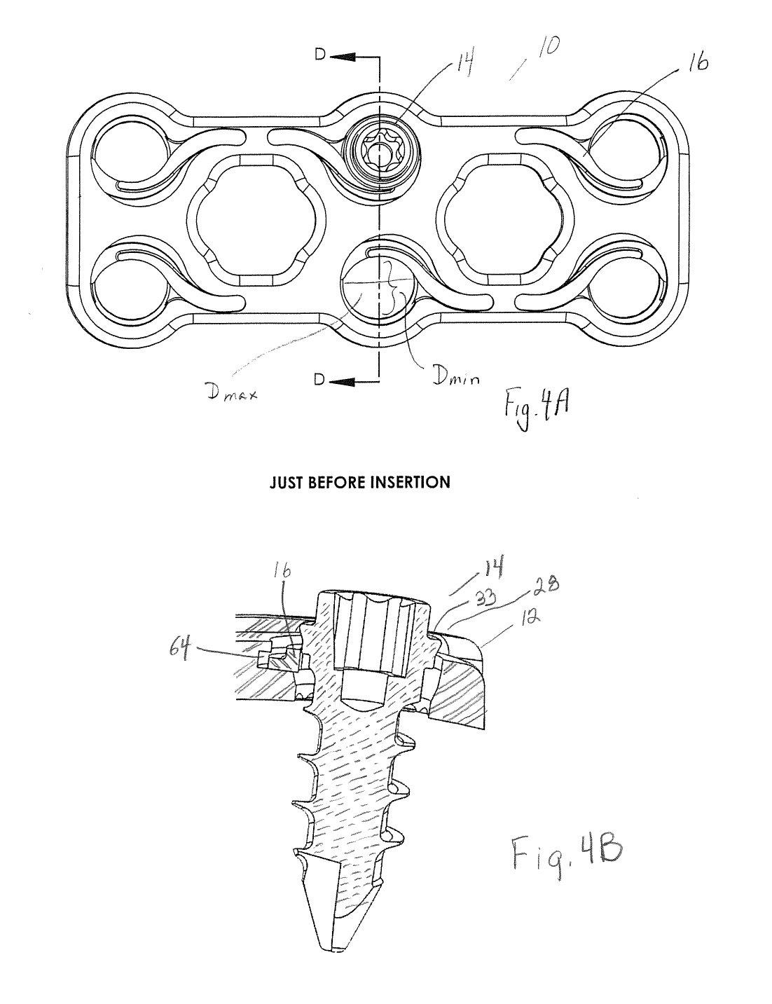

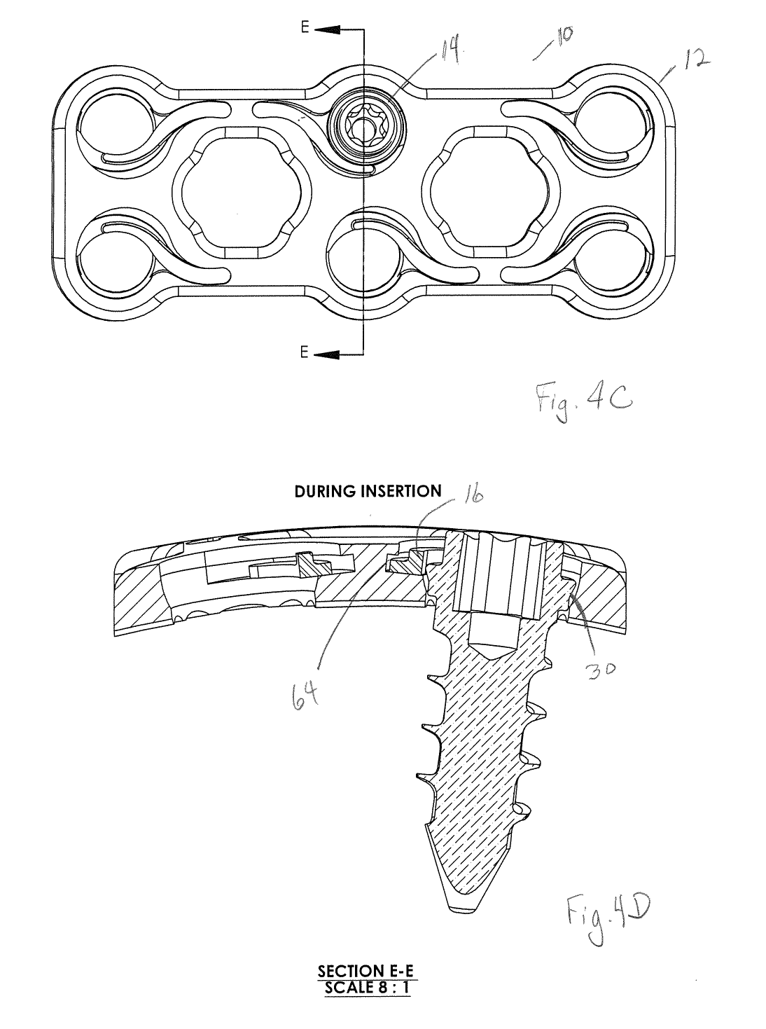

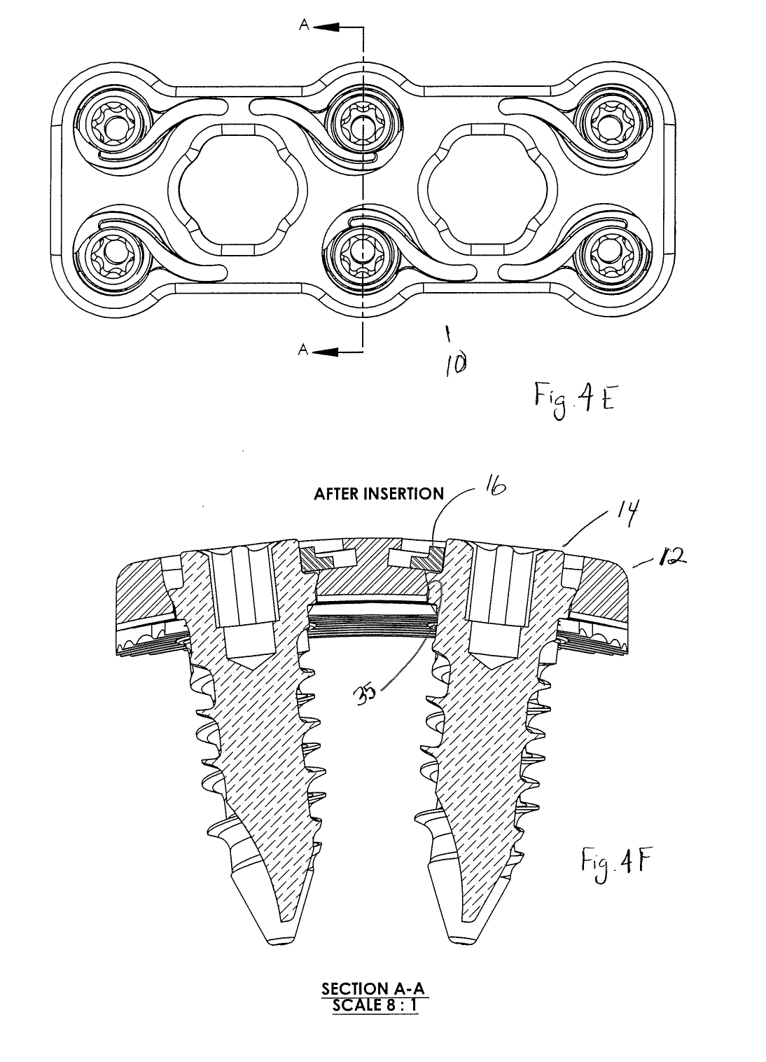

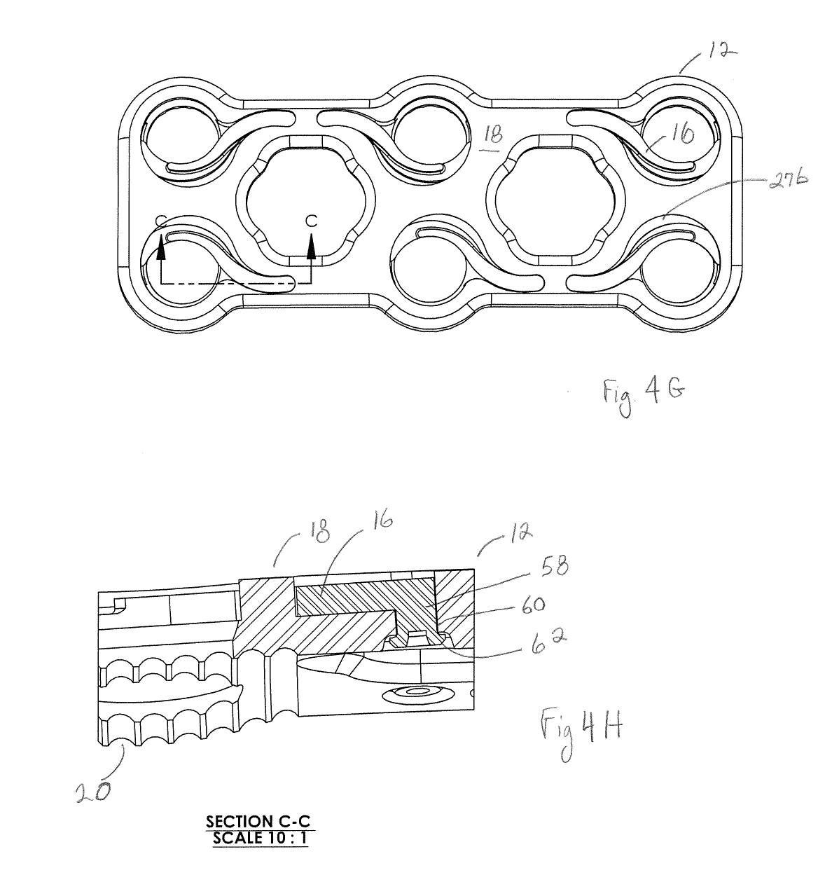

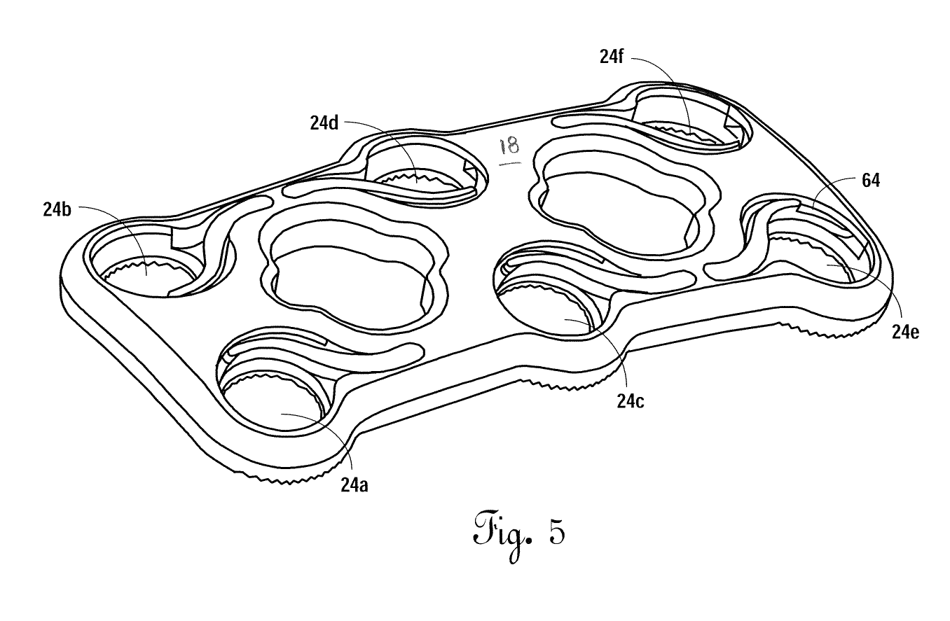

Turning back to stepped section 29 and to the general shape of the screw as seen in FIGS. 1, 1A, 2, and 4, the screw may be seen to have a first diameter at D1 (at side walls 31), second diameter greater than the first at D2 (widest point, conical section walls meet head floor 33), and a third diameter at D3, where the head (at lower end of conical section 35) joins the tapered threaded section 36 or vertical walls 31a (on fixed pitch screw). In one embodiment, D1 is about equal or greater than D3 (see FIG. 2A), and D1 is smaller than D2. FIG. 1A shows the height of the screw head and FIG. 2C the height of screw bore 24a. The screw head is dimensioned to fit snugly in the screw bore, in one embodiment (see FIG. 4), with the retainer arm body in a locking position.

Turning now to FIG. 2A, it is seen, in one embodiment, that the overall shape of Applicant's arm 16 may be S-shaped or a “lazy S”. In either case, arm 16 may have a tail section 40 at one end thereof and a curved head engaging or collar section 48 near the other end thereof, and a free or flex section 46 therebetween with a ‘pivot” or flex area A (see FIGS. 1 and 4). There may also be stop wall engaging section 42 (see FIG. 1) to the S-shaped arm. In one embodiment, a backbone or thickened section 51 may be provided from the beginning of the tail section to about three-quarter of the way to the removed end to provide strength, so the arm will not break or deform during flexing. Arm 16 typically has a flat upper surface 50 (upper surface of the backbone) and a flat lower surface 52, which lays adjacent retainer arm floor 27b. Perimeter side walls 54 separate the upper surface from the lower surface of the arm. An intermediate surface 53 between surfaces 50 and 52 represents the upper surface of the non-backbone part or pocket engaging section 56 of arm 16, which is dimensioned to be received at least party into undercut or pocket 64.

FIG. 1A shows minimum maximum diameter of the screw head D2 and the minimum screw diameter D3. Turning to FIGS. 1A and 4A, a Dmin and a Dmax are seen when the retainer arm 16 partially covers the upper diameter of the screw bore. See FIG. 2C Duo. In one embodiment, the minimum diameter of the screw head is less than Dmin of FIG. 4A. In another embodiment, the screw head minimum diameter is between Dmin and Dmax of FIG. 4A. In a preferred embodiment, the maximum diameter of the screw head is greater than Dmin in 4A and less than Duo, the upper diameter of the screw bore as seen in FIG. 2C.

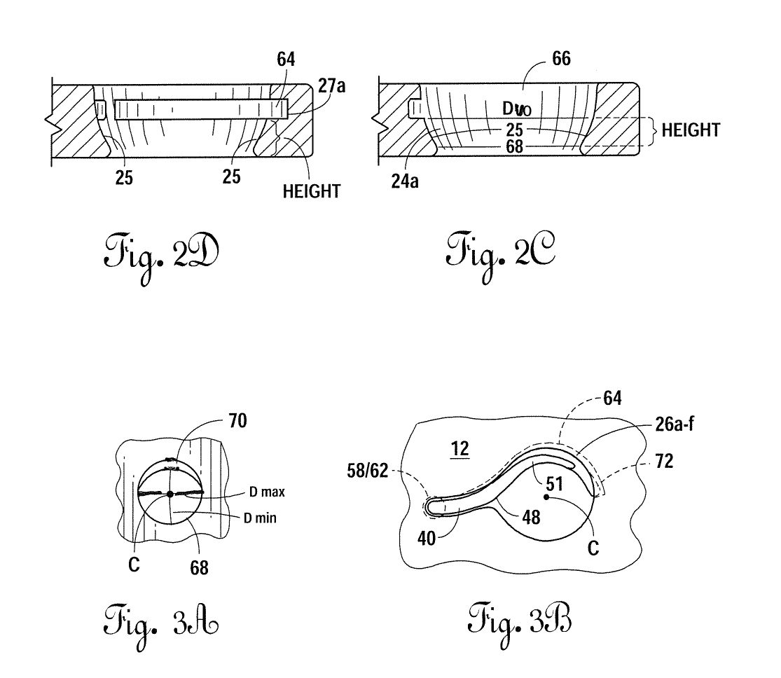

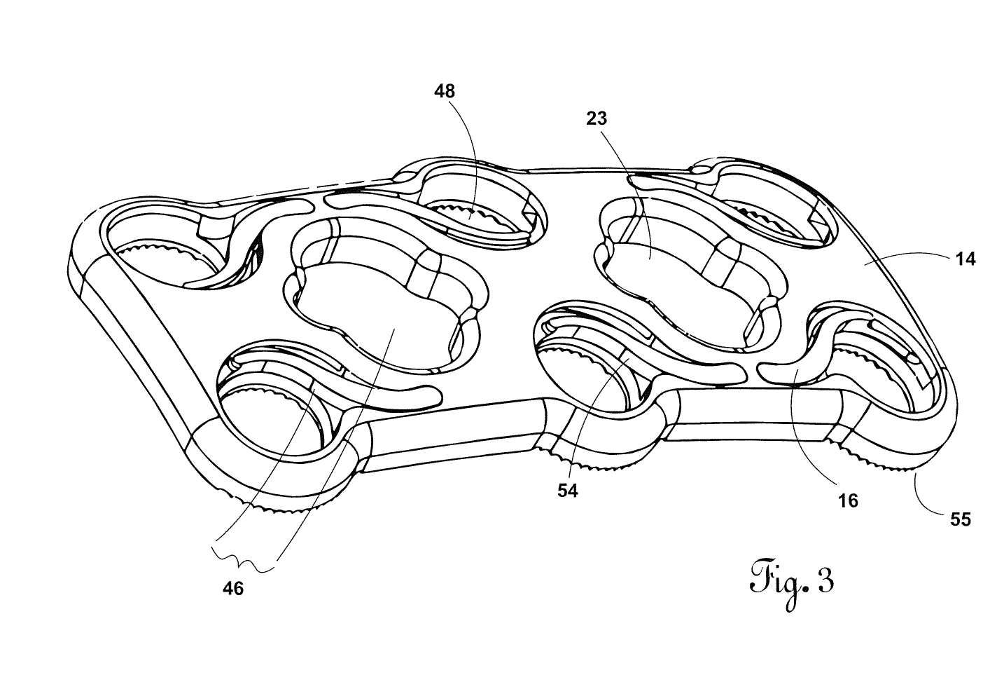

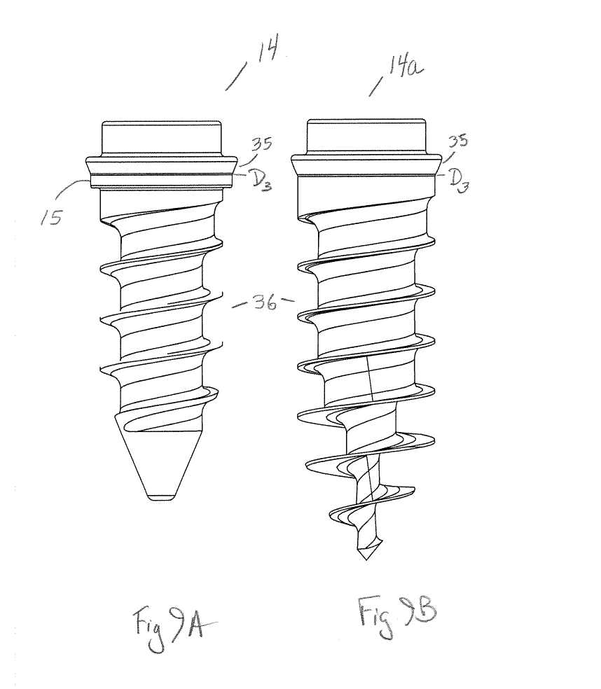

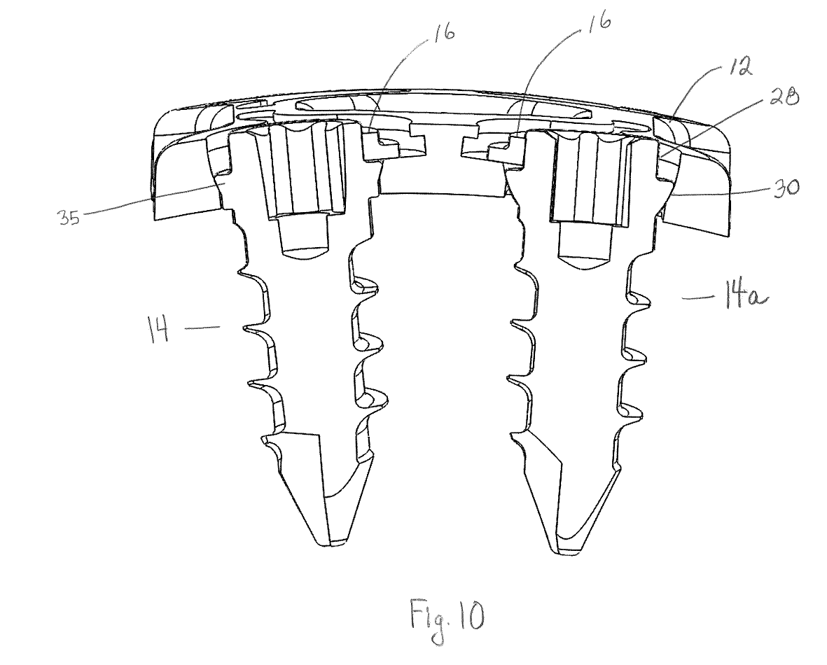

FIGS. 9A, 9B, and 10 illustrate a fixed pitch 14 and a variable pitch 14a screw, with FIG. 10 illustrating the screws as they sit in a plate having a screw bore. The term fixed and variable pitch does not mean pitch of the threader of the screw, but it means that the fixed pitch when the screw is locked down and seated in the screw bore has almost no and (typically less than 1 degree) of movement of the threaded shank. That is to say, the fixed pitch has very little movement and the surgeon does not have a lot of angular range around center C (see FIGS. 3B and 4F) when inserting the screw into the screw bore. The variable pitch, however, which does not have the vertical cylindrical depending walls 31a beneath the lower diameter D3 of the head as is found in FIG. 9A, in one embodiment, allows up to about 15 degrees angular movement about a longitudinal axis, thereby allowing the surgeon to place the screw at a variety of angles. In one embodiment of the variable pitch screw, the difference between the minimum diameter of the screw head and the lower diameter of the screw bore may be up to about 10-12 mil. allowing the up to 15 degree pivotal movement. Note the screw tips in FIG. 4 are sharp self-drilling and the screw tips in FIG. 10 are blunt (self-tapping).

Turning to FIGS. 4A-4G, in one embodiment of the fixed pitch screw 14, the diameter D2 and the diameter Rc (see FIG. 2A) of head engaging collar section 48 of arm 16 are typically about equal or Rc may be slightly larger. There is a flex point at A and a “free” section 46, such that before the screw advances into the screw bore, at least part of collar section 48 extends into the screw bore (see FIG. 4A) a distance insufficient to engage the threaded section of the screw, but sufficient to catch the screw head side walls 30 below D2. By the time there is engagement of collar section 48 to the side walls 30 (see FIG. 4), the screws have a good bite on the bone and are advancing thereinto. As the screws advance into the bone, the arm will flex at point or area A, primarily along the free section 46. As the screw advances and forces the arm to flex outward, it will come to a point where the screw head floor 33 will pass the arm at D2, the uppermost diameter of conical section 35, and (the arm being resilient) snap into locking engagement over the top of the screw head floor. Interference between the screw head seating in the screw bore may prevent further engagement of the screw into the bone and provide the surgeon with a positive stop. In one embodiment, the screw is prevented from backing out, in part, by the seated or locked arm whose tension at flex point or area A and across the free section will retain the spring in place near or against side walls 31 of stepped back section 29. In another embodiment, there is no stepped back section, just head floor 33 with a tool engaging section extending down into. In that case, the arm extending partly into undercut portion 64 helps prevent backout if a screw starts to back out and urges the underside of the arm upward towards the upper surface of the plate. A suitable material for making this spring is tungsten or any other suitable material capable of flexing without breaking.

FIGS. 2A and 6 illustrate that tail section 40 may include a pin or rivet 58 extending downward through a bore 60, which bore 60 passes all or partly through the plate. An annular lip 62 at the end of the pin or rivet 58 will lock the resilient clip, especially the tail, into a position such that it does not lift out of the retainer arm recesses 26a/26b/26c/26d/26e/26f. Collar section 48 may also be held in place using a collar pocket or undercut portion 64, representing an arcuate recess in the arm retainer recesses 26a/26b/26c/26d/26e/26f for receiving at least some of pocket engaging section 56 of the arm. The arm is configured such that at least part of section 56 may retreat into pocket 64 while the screw head is pushing collar section 48 outward (screw being inserted into bone). Moreover, the width of section 56 and the depth of pocket 64 are such that, when the screw is seated with the arm in a locked in position (see FIG. 4), the upper wall of pocket 64 will overhang some of the outer edge of section 56 (see Detail A FIG. 4), so that there is interference should a screw attempt to back out, that is to say, interference between section 56 and the lip overhang defining the upper walls of pocket 64 (see also FIG. 4 Detail A).

FIG. 4 also illustrates one way in which screw bore side walls 25 engage head side walls 30, here, with their contours or profiles generally matching, but with some “play” (in the variable pitch embodiment), to provide some poly axial movement of the screw when the walls are so configured. The screws may also be fixed (tight) in another embodiment (fixed pitch). Moreover, the threaded section of the screws may be self-drilling or self-tapping. The stepped back distance Sd, which may also be the difference between D2 and D3, may be in one preferred embodiment about 20 mil.

In summary, the arm, in one embodiment, has a generally “S” shape. At the tail section 40, it engages the plate 12 removed from the tail is a screw head engaging collar section 48 with inner walls, in one embodiment, having a diameter Rc between about D2 and D3, and which collar section 48 may have an outer pocket engaging section 56. Between the collar section 48 and tail section 40 is a free or flex section 46 that is typically not engaged with the plate (except at its lower surface), either when the spring is fully under flex (just before the advancing screw upper edge of conical section 35 passes the lower edge of conical section 35) and in the locked position, just after.

In FIGS. 2C, 2D, 3A, 3B, 4A and 6, a cross-section top and bottom views of the plate are seen. Screw bore lower opening 68 has a diameter sufficiently large to allow the threaded section to pass and smaller than D2 and at or slightly larger than D3 to allow the screw to seat, but still allow some “play” (variable pitch) with the screw longitudinal axis. FIG. 4B shows a top view of an arm 16 in the arm retainer recess 26a/26b/26c/26d/26e/26f (no screw). The dashed lines showing the pocket or undercut portion 64 of the recess that undercuts the surface to allow the arm to flex as the screw is inserted into the screw bore. A nose portion 72 configured to engage the undercut at least in the unflexed (relaxed) positions of the arm viewed in FIGS. 4A and 4B which position of the arm is typically substantially the same the locked position FIGS. 4E and 4F.

One of the materials comprising the assembly may be anodized titanium. Anodized titanium may be available in a number of colors. Typically, arm 16 will be in a color, such as green, contrasting to that of plate 12, which may be gray, and screw 14, which may be gold colored. Contrasting colors, especially between the screw and the arm, is important for good visibility, namely, location of the arm relative to the screw.

See-through window or graft window 23 allows the surgeon to see the graft. In a preferred embodiment of Applicant's plate, the graft window 23 has what may be referred to as a “club/clover” shape. The smaller diameter curved sections extend into the plate portion where the screws are closer to one another (lateral spacing) and the larger diameter portions of the club/clover shape extend into the plate portions where the screw spacing tends to be longer (longitudinal spacing). A one-level assembly would typically have four screws; two level plate, six screws; three level plate having eight screws. A multiplicity of serrated knife edge ribs 55 (see FIGS. 4 and 6) may be provided on the underside of the plate for better grip, plate to bone.

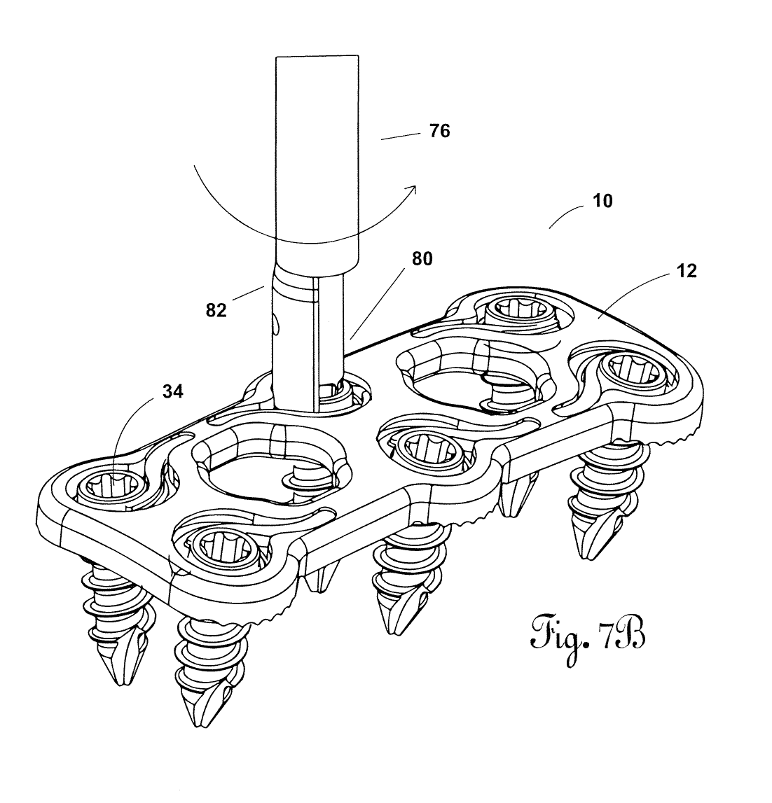

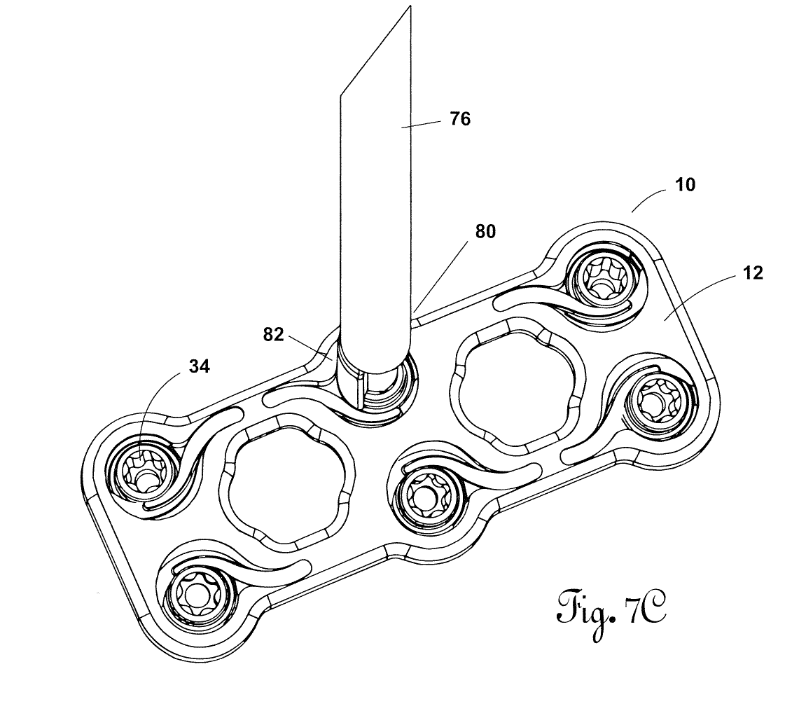

FIGS. 7A, 7B, and 7C illustrate that assembly 10 may include, in one embodiment, a screw driving tool 76. Tool driving tool 76 may have a handle portion 78 adapted to be grasped with a hand, and may be cylindrical, and a nose portion 80, which may be configured to engage tool engaging walls 34 of screws 14. A cup extension 82 extends downward from the handle to lay adjacent the head screw removal tool has the cup shape extension such that rotation will allow the removed end of the cup shape extension to push the collar section of the screw out of an interference position with the screw head. For driving the screw into the bone, through the plate into the seated position with the clips locking the screw head in place, one uses tool 78 without cup extension 82 thereon.

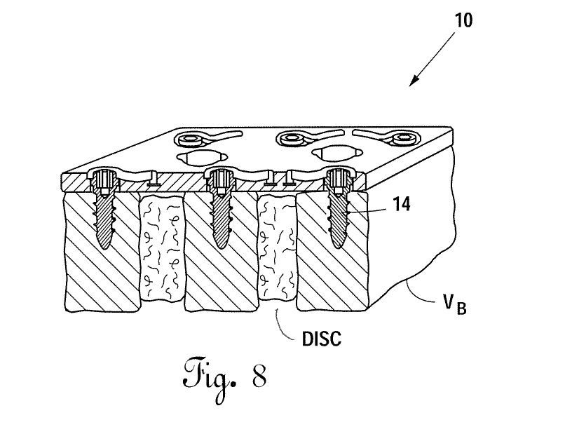

FIG. 8 shows a three level plate assembly engaging vertebrae.

Although the invention has been described with reference to a specific embodiment, this description is not meant to be construed in a limiting sense. On the contrary, various modifications of the disclosed embodiments will become apparent to those skilled in the art upon reference to the description of the invention. It is therefore contemplated that the appended claims will cover such modifications, alternatives, and equivalents that fall within the true spirit and scope of the invention.

1. A bone fixation assembly comprising:
a multiplicity of screws, each having a threaded section with a tip at a first end and a head having an upper surface at a second end, the head having a first diameter where it meets the threaded section and a larger diameter at the upper surface; and a head height between the two diameters;
a plate having a plane and having an upper surface, a lower surface, and a multiplicity of screw bores therethrough, the screw bores having an upper opening having a first diameter and a lower opening having a second diameter and a screw bore height between the two diameters, the plate having a retainer arm recess floor defining a retainer arm recess floor plane, the retainer arm recess floor above the walls defining the screw bore, the plate having a multiplicity of pin bores therethrough, such that each screw bore has a pin bore adjacent thereto; and
a single retainer arm with a first end having a depending pin for engaging the pin bore of the plate adjacent each screw bore and a removed end with a body therebetween, the removed end not attached to the plate and free to move in the plane of the retainer arm recess floor such that in a first position part of the retainer arm body blocks some of the upper opening of the screw bore and in a second position, responsive to contact with a screw head as the screw head rotates and moves longitudinally into a screw bore, moves to a second, loaded position outward from the first position, and with further longitudinal movement of the screw such that the upper surface of the screw head passes below the upper opening of the screw bore, substantially returns to the first position.

2. The bone fixation assembly of claim 1, wherein the retainer arm recess floor is below the upper surface of the plate and wherein the plate includes a pocket for each screw bore dimensioned to receive at least part of the resilient retainer arm, the pockets below the upper surface of the plate and above the screw bores.

3. The bone fixation assembly of claim 2, wherein the pocket is dimensioned to receive part of the retainer arm when the retainer arm is moving towards and away from the loaded position.

4. The bone fixation assembly of claim 1, wherein the retainer arm is resilient and includes a lower surface which lays against the retainer arm recess floor and an intermediate surface between the upper and lower surface.

5. The bone fixation assembly of claim 4, wherein the retainer arm recess floor is below the upper surface of the plate and wherein the plate includes a pocket for each screw bore dimensioned to receive at least part of the resilient retainer arm, the pockets below the upper surface of the plate and above the screw bores.

6. The bone fixation assembly of claim 1, wherein the retainer arm has an “S” shape.

7. The bone fixation assembly of claim 1, wherein the plate includes upstanding walls adjacent the first end of the retainer arm, which upstanding walls are dimensioned to contact retainer arm walls adjacent the first end of the retainer arm while the retainer arm is moving between the two positions, but not when the retainer arm is in the first position.

8. The bone fixation assembly of claim 1, wherein the plate, retainer arm, and screw are all in contrasting colors.

9. The bone fixation assembly of claim 1, wherein the screw head includes tool engaging walls.

10. The bone fixation assembly of claim 8, wherein the tool engaging walls project upward from the upper surface of the screw head.

11. The bone fixation assembly of claim 1, wherein below the first diameter of the screw head, there are cylindrical walls projecting perpendicular downward from the screw head with a diameter just slightly smaller than the second diameter of the plate, to substantially prevent pitch movement of the screw when it is seated in the screw bore.

12. The bone fixation assembly of claim 1, wherein the screw is a variable pitch screw wherein conical walls of the head meet the threaded section of the screw.

13. The bone fixation assembly of claim 1, wherein the screw head first diameter is substantially smaller than the second diameter of the screw head.

14. A bone fixation assembly comprising:
a multiplicity of screws, each having a threaded shank and a head with a conical portion and a screw head top surface, the head having a first diameter wherein the conical section meets the screw head top surface and a second smaller diameter wherein the head meets the threaded shank;
a multiplicity of “S” shaped resilient retainer arms, the multiplicity of retainer arms equaling the number of multiplicity of screws, each retainer arm with a tail section, the tail section having a near end and a screw head engaging section, the screw head engaging section having inner walls and a lower surface; and
a plate having a top surface, a bottom surface and an outer perimeter, the plate having a multiplicity of screw bores each having an upper diameter dimensioned to allow the screw head to pass through and a smaller, lower diameter, the plate including a retainer arm recess portion for each screw, each retainer arm recess portion with a retainer arm floor and with sidewalls, the screw bores having screw bore sidewalls configured to engage at least part of the conical portion of the screw head when the screw is in a seated and locked down position with the screw head engaging section of the resilient retainer arm overlaying a portion of the screw head top surface, the screw bore configured to receive screws such that the threaded section of the screw passes through the plate and the head seats at least partly in the screw bore sidewalls;
wherein the plate has a retainer through bore opening in the retainer arm floor; and
wherein each retainer arm has a depending member from the tail section adapted to pivotally engage the plate at the retainer through bore opening .

15. The bone fixation assembly of claim 14, wherein below the second diameter of the screw head, there are cylindrical walls projecting perpendicular downward with a diameter just slightly smaller than the lower diameter of the screw bore to substantially prevent pitch movement of the screw when it is seated and locked down in the screw bore.

16. The bone fixation assembly of claim 14, wherein the screw is a variable pitch screw and wherein the conical walls of the head meet the threaded section of the screw.

17. The bone fixation assembly of claim 14, wherein the retainer arm floor is below the upper surface of the plate and wherein the plate includes a pocket for each screw bore dimensioned to receive at least part of the resilient retainer arm, the pockets below the upper surface of the plate and above the screw bores.

18. The bone fixation assembly of claim 17, wherein the pocket is dimensioned to receive part of the resilient retainer arm when the resilient retainer arm is moving away from the position it is in when the screw is in the seated and locked down position.

19. The bone fixation assembly of claim 14, wherein the plate, retainer arm, and screw are all in contrasting colors.
